(19) United States
(12) Patent Application Publication
(10) Pub. No.: US 2025/0314570 A1
(43) Pub. Date: Oct. 9, 2025

(54) FOLDING INSPECTION APPARATUS

(71) Applicant: Samsung Display Co., LTD., Yongin-si (KR)

(72) Inventors: JUNGHOON OH, Yongin-si (KR); KITAEK KIM, Yongin-si (KR); SAEROM KIM, Yongin-si (KR); KYUNGEUN LEE, Yongin-si (KR)

(21) Appl. No.: 19/051,868

(22) Filed: Feb. 12, 2025

(30) Foreign Application Priority Data
Apr. 9, 2024 (KR) 10-2024-0047769

(51) Int. Cl. G01N 3/20; G01N 3/04; G01N 33/38

(52) U.S. Cl. CPC G01N 3/20; G01N 3/04; G01N 33/386

(57) ABSTRACT

A folding inspection apparatus includes a first plate on which a foldable thin-film glass is disposed, a second plate which faces the first plate in a first direction, and on which the thin-film glass is disposed, a first fixing member which is disposed on an end of the first plate, and in which first grooves are defined, a second fixing member which is disposed on an end of the second plate, and in which second grooves respectively corresponding to the first grooves in the first direction are defined, a first fixing block coupled to a part of the first grooves, and contacting at least one outer surface of the thin-film glass, and a second fixing block coupled to a part of the second grooves, and contacting at least one outer surface of the thin-film glass. The first and second fixing blocks do not overlap each other in the first direction.

This application claims priority to Korean Patent Application No. 10-2024-0047769, filed on Apr. 9, 2024, and all the benefits accruing therefrom under U.S.C. § 119, the content of which in its entirety is herein incorporated by reference.

### BACKGROUND

### 1. Field

The disclosure herein relates to a folding inspection apparatus, and more particularly, to a folding inspection apparatus for thin-film glass.

### 2. Description of the Related Art

A display device includes a display module including a window and a display panel. The display panel may include various elements, such as a display element, a touch element, a detection element, or the like, which is activated in response to an electrical signal.

The window protects the display panel, and provides an active region to a user. Accordingly, a user provides an input to the display panel or receives information generated in the display panel, through the window. In addition, the display panel may be stably protected from an external impact through the window.

Recently, folding properties of the display module are becoming significant due to a trend of providing the slimness of a display device and the extensibility of a display region.

### SUMMARY

The disclosure provides a folding inspection apparatus which prevents lowering of inspection reliability caused by distortion of a thin-film glass when the thin-film glass is folded and the flexural strength of the thin-film glass is measured.

The disclosure also provides a folding inspection apparatus with which it is possible to reduce a time desired for inspection since it is unnecessary to separately inspect crack lines of a thin-film glass.

An embodiment of the inventive concept provides a folding inspection apparatus including a first plate on which a foldable thin-film glass is seated, a second plate which faces the first plate in a first direction, and on which the foldable thin-film glass is disposed, a first fixing member which is disposed on an end of the first plate, and in which a plurality of first grooves is defined, a second fixing member which is disposed on an end of the second plate, and in which a plurality of second grooves respectively corresponding to the plurality of first grooves in the first direction is defined, a first fixing block coupled to a part of the plurality of first grooves, and contacting at least one outer surface among outer surfaces of the foldable thin-film glass; and a second fixing block coupled to a part of the plurality of second grooves, and contacting at least one outer surface among outer surfaces of the foldable thin-film glass, wherein the first fixing block and the second fixing block do not overlap each other in the first direction.

In an embodiment, the first fixing block and the second fixing block may each be provided in plural, and a plurality of first fixing blocks and a plurality of second fixing blocks may be alternately disposed in a second direction crossing the first direction and in a third direction crossing the second direction.

In an embodiment, a first thickness of the first fixing block along the first direction and a second thickness of the second fixing block along the first direction may be greater than a thickness of the foldable thin-film glass along the first direction.

In an embodiment, the plurality of first grooves may include a (1-1)-th groove extending in a second direction crossing the first direction, and a (1-2)-th groove extending in a third direction crossing the second direction, the plurality of second grooves may include a (2-1)-th groove extending in the second direction, and a (2-2)-th groove extending in the third direction, and the (1-1)-th groove may correspond to the (2-1)-th groove in the first direction, and the (1-2)-th groove may correspond to the (2-2)-th groove in the first direction.

In an embodiment, the (1-1)-th groove may be provided in plural, at least one (1-1)-th groove of a plurality of (1-1)-th grooves may be extended to the (1-2)-th groove, the (2-1)-th groove may be provided in plural, and at least one (2-1)-th groove of a plurality of (2-1)-th grooves may be extended to the (2-2)-th groove.

In an embodiment, the (1-1)-th groove and the (2-1)-th groove may each be provided in plural, a plurality of (1-1)-th grooves may include a first row in which (1-1)-th grooves among the plurality of (1-1)-th grooves are arranged along the second direction, and a second row in which (1-1)-th grooves among the plurality of (1-1)-th grooves are arranged along the second direction and which is spaced apart from the first row along the third direction, and a plurality of (2-1)-th grooves may include a third row in which (2-1)-th grooves among the plurality of (2-1)-th grooves are arranged along the second direction, and a fourth row in which (2-1)-th grooves among the plurality of (2-1)-th grooves are arranged along the second direction and which is spaced apart from the third row along the third direction.

In an embodiment, the (1-2)-th groove and the (2-2)-th groove may each be provided in plural, and a plurality of (1-2)-th grooves may be arranged along the third direction, and a plurality of (2-2)-th grooves may be arranged along the third direction.

In an embodiment, the first fixing block may be provided in plural, a plurality of first fixing blocks may include a (1-1)-th fixing block coupled to the (1-1)-th groove, and a (1-2)-th fixing block coupled to the (1-2)-th groove, the second fixing block may be provided in plural, and a plurality of second fixing blocks may include a (2-1)-th fixing block coupled to the (2-1)-th groove, and a (2-2)-th fixing block coupled to the (2-2)-th groove.

In an embodiment, a minimum distance along the second direction between the plurality of first fixing blocks and the plurality of second fixing blocks may be about 5 mm or more.

In an embodiment, the (1-1)-th fixing block and the (2-1)-th fixing block may contact a first outer surface, which extends in the second direction, among the outer surfaces of the foldable thin-film glass, and the (1-2)-th fixing block and the (2-2)-th fixing block may contact a second outer surface, which extends in the third direction, among the outer surfaces of the foldable thin-film glass.

In an embodiment, the foldable thin-film glass disposed on the first plate and the second plate may include a folded portion of which a curvature becomes greater as the first plate and the second plate get closer to each other.

In an embodiment, the first fixing member may correspond to the second fixing member in the first direction.

In an embodiment, the foldable thin-film glass may include a first portion disposed on the first plate, a second portion disposed on the second plate, and a folding portion connected to the first portion and the second portion, and foldable, the first fixing block may fix a terminal end of the first portion, and the second fixing block may fix a terminal end of the second portion.

In an embodiment, a thickness of the foldable thin-film glass may be about 100 micrometers or less.

In an embodiment, the first fixing member may be detachably coupled to the first plate, and the second fixing member may be detachably coupled to the second plate.

In an embodiment, the first fixing block may be detachably coupled to the first fixing member, and the second fixing block may be detachably coupled to the second fixing member.

In an embodiment of the inventive concept, a folding inspection apparatus includes a first plate on which a foldable thin-film glass is seated, a second plate which faces the first plate in a first direction, and on which the foldable thin-film glass is disposed, a first fixing member which is disposed on an end of the first plate, and in which a plurality of first grooves are defined, a second fixing member which is disposed on an end of the second plate, and in which a plurality of second grooves respectively corresponding to the plurality of first grooves in the first direction are defined, a first fixing block coupled to a part of the plurality of first grooves, and contacting at least one outer surface among outer surfaces of the foldable thin-film glass, and a second fixing block coupled to a part of the plurality of second grooves, and contacting at least one outer surface among the outer surfaces of the foldable thin-film glass, wherein the first fixing block and the second fixing block are each provided in plural, and a plurality of first fixing blocks and a plurality of second fixing blocks are alternately disposed along a second direction crossing the first direction and along a third direction crossing the second direction.

In an embodiment, a first thickness of the first fixing block along the first direction and a second thickness of the second fixing block along the first direction may be greater than a thickness of the foldable thin-film glass along the first direction.

In an embodiment, a minimum distance along the second direction between the plurality of first fixing blocks and the plurality of second fixing blocks may be about 5 mm or more.

In an embodiment, the foldable thin-film glass may include a first portion disposed on the first plate, a second portion disposed on the second plate, and a folding portion connected to the first portion and the second portion, and foldable, the first fixing block may fix a terminal end of the first portion, and the second fixing block may fix a terminal end of the second portion.

### BRIEF DESCRIPTION OF THE DRAWINGS

The accompanying drawings are included to provide a further understanding of the inventive concept, and are incorporated in and constitute a part of this specification. The drawings illustrate embodiments of the inventive concept and, together with the description, serve to explain principles of the inventive concept.

### IN THE DRAWINGS

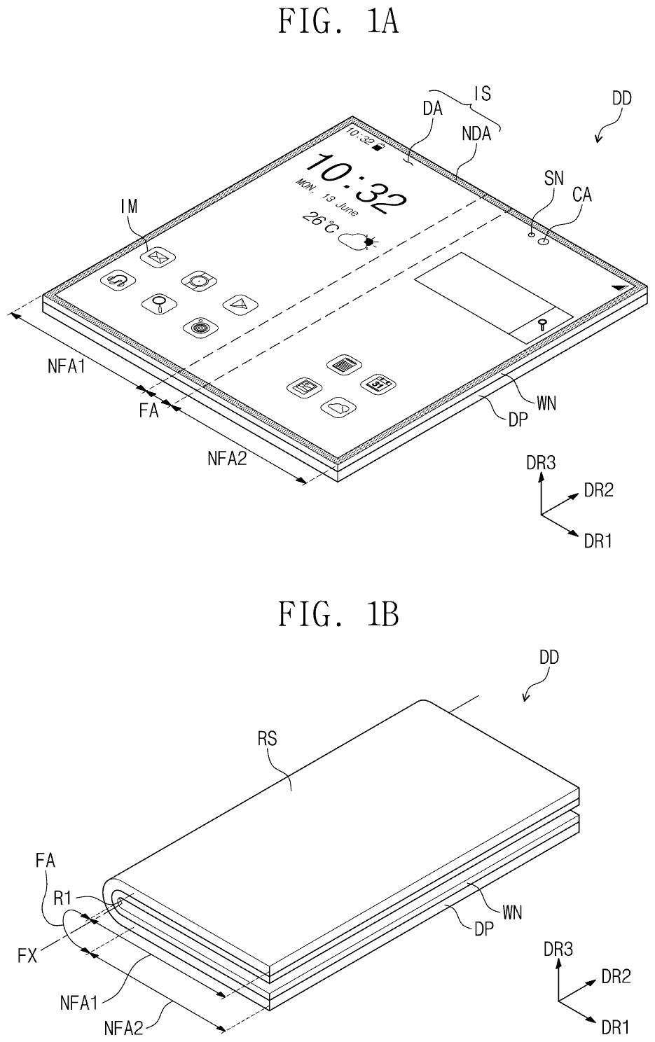

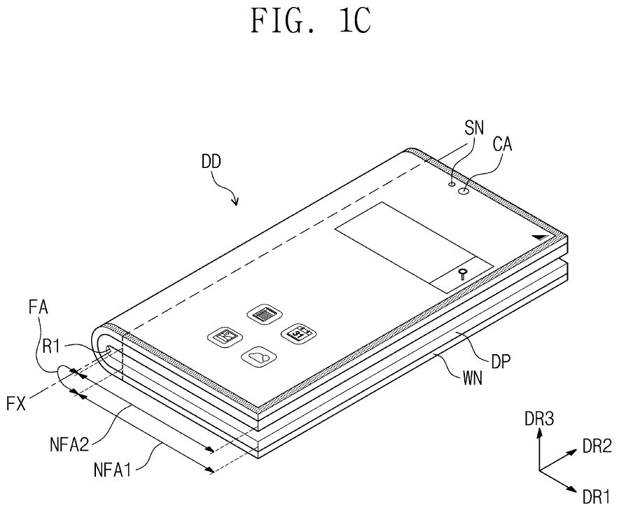

FIGS. 1A to 1C are perspective views of an embodiment of a display device according to the inventive concept;

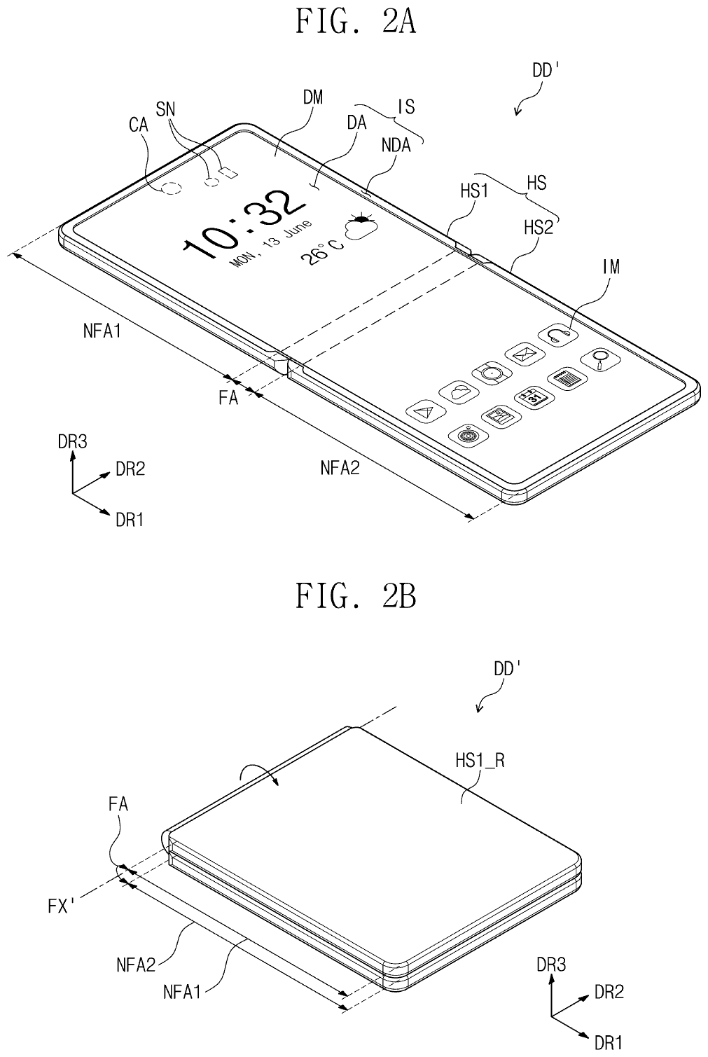

FIGS. 2A and 2B are perspective views of another embodiment of a display device according to the inventive concept;

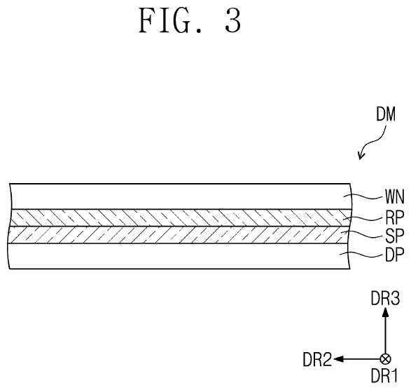

FIG. 3 is a cross-sectional view of an embodiment of a display module according to the inventive concept;

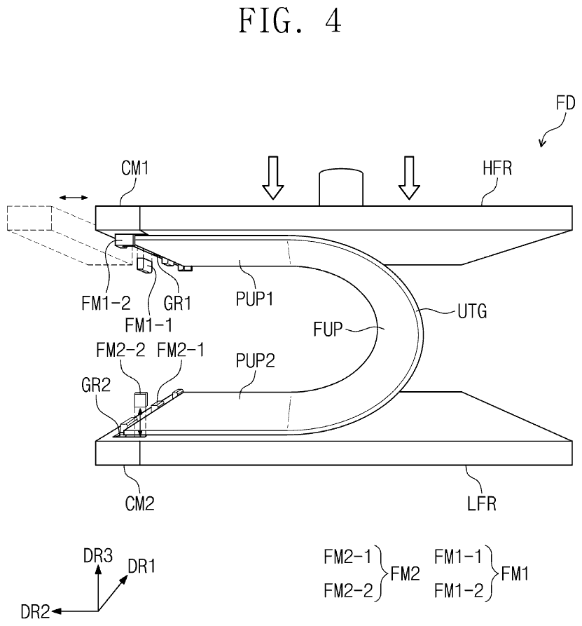

FIG. 4 is a perspective view of an embodiment of a folding inspection apparatus according to the inventive concept;

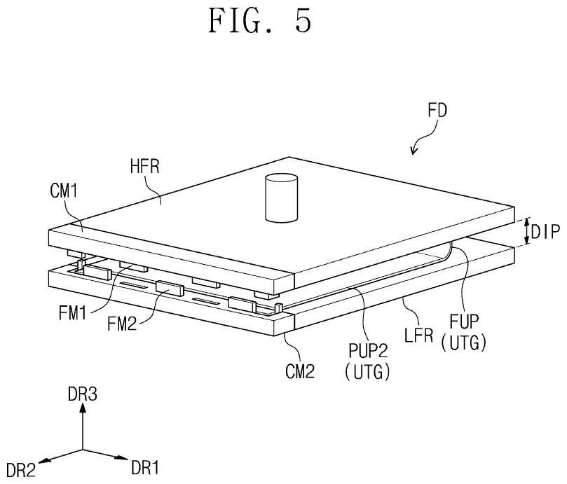

FIG. 5 is a perspective view of an embodiment of a folding inspection apparatus according to the inventive concept;

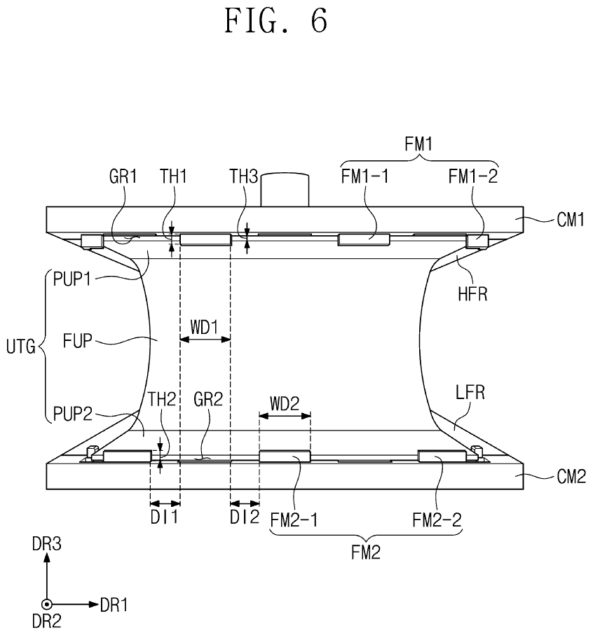

FIG. 6 is a front perspective view of an embodiment of a folding inspection apparatus according to the inventive concept;

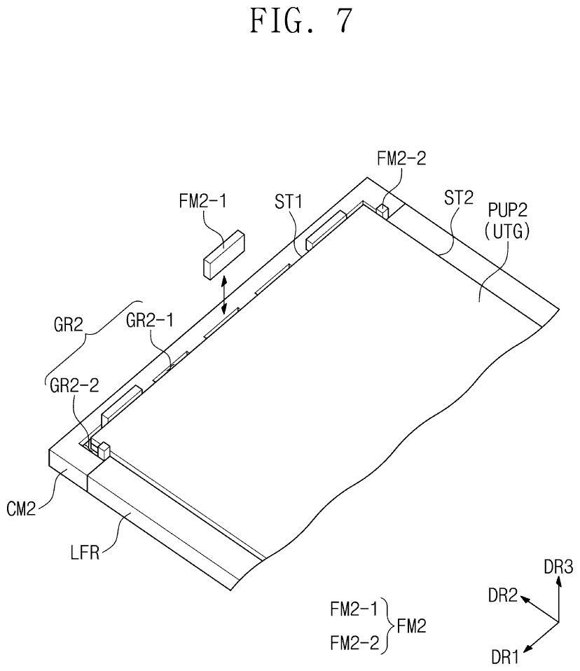

FIG. 7 is a perspective view of an embodiment of a second fixing member and a second plate of a folding inspection apparatus according to the inventive concept;

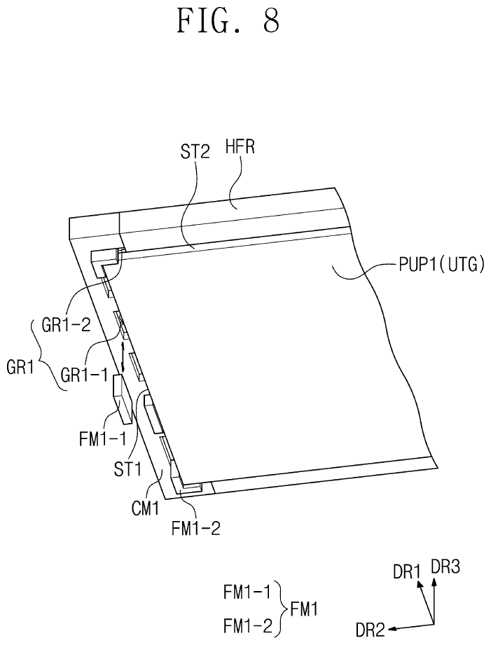

FIG. 8 is a perspective view of an embodiment of a first fixing member and a first plate of a folding inspection apparatus according to the inventive concept;

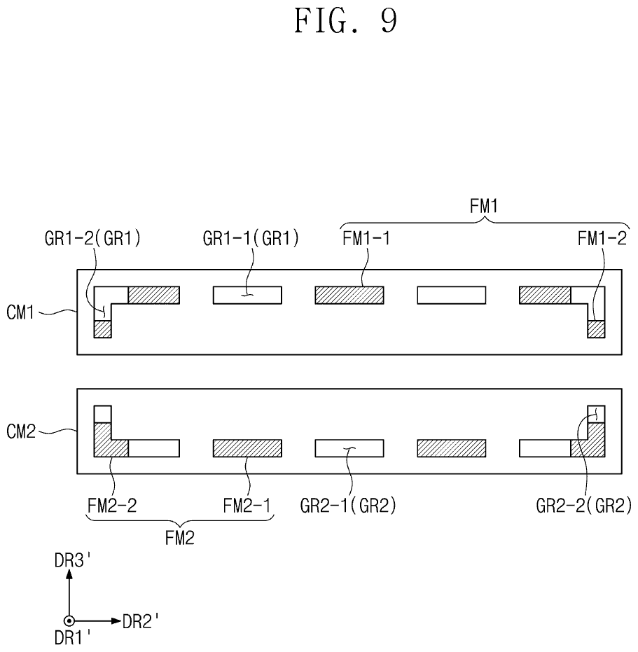

FIG. 9 is a plan view of an embodiment of a first fixing member and a second fixing member according to the inventive concept;

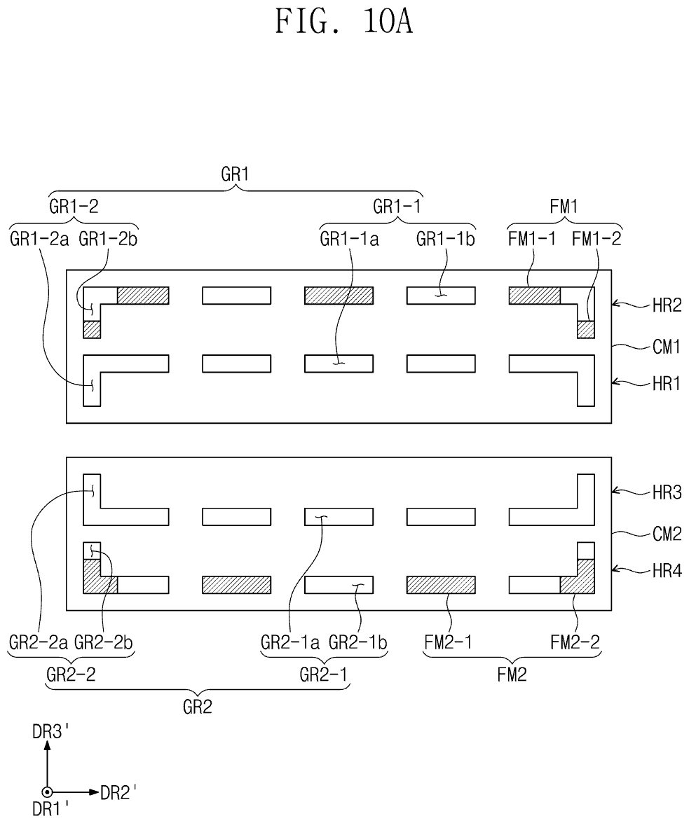

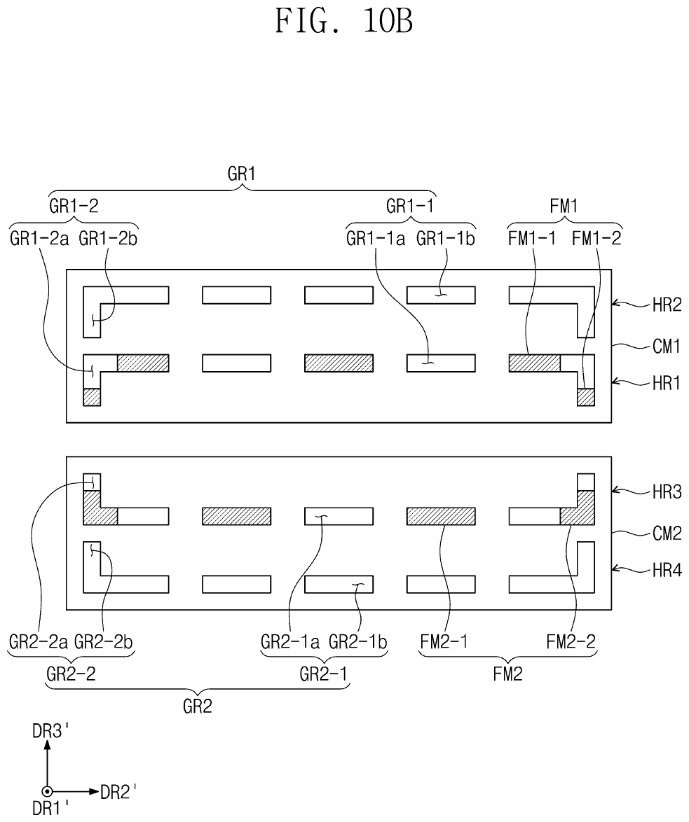

FIGS. 10A and 10B are plan views of another embodiment of a first fixing member and a second fixing member according to the inventive concept; and

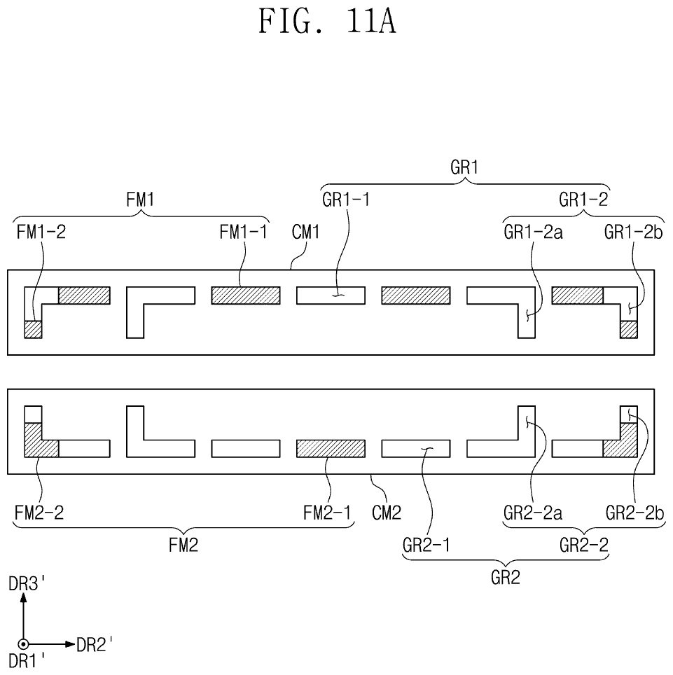

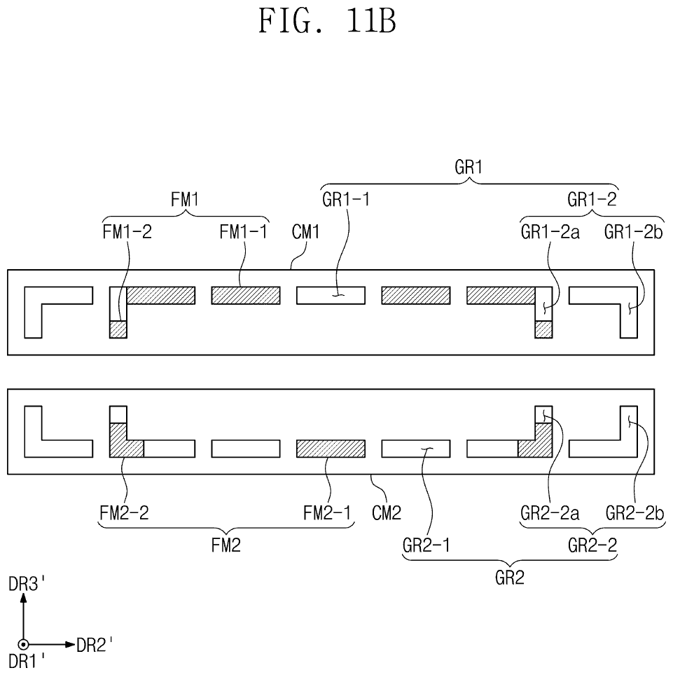

FIGS. 11A and 11B are plan views of another embodiment of a first fixing member and a second fixing member according to the inventive concept.

### DETAILED DESCRIPTION

In this specification, it will be understood that when an element (or region, layer, portion, etc.) is referred to as being “on”, “connected to” or “coupled to” another element or layer, it may be directly disposed on/connected to/coupled to the other element or intervening elements may be present.

Like reference numerals or symbols refer to like elements throughout the specification. In addition, in terms of drawings, the thickness and the ratio and the dimension of the element are exaggerated for effective description of the technical contents. As used herein, the term “and/or” includes any and all combinations of one or more of the associated listed items.

Terms such as first and second may be used to describe various components, but the components should not be limited by the terms. These terms are only used for the purpose of distinguishing one component from other components. For example, without departing from the scope of the inventive concept, a first component may be referred to as a second component, and similarly, a second component may be referred to as a first component. Singular expressions include plural expressions unless the context clearly indicates otherwise.

Spatially relative terms, such as “beneath”, “below”, “lower”, “above”, “upper” and the like, may be used herein for ease of description to describe one element or feature's relationship to another element(s) or feature(s) as illustrated in the drawing figures. It will be understood that the spatially relative terms are intended to encompass different orientations of the device in use or operation in addition to the orientation depicted in the drawing figures.

It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which the disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, embodiments of the inventive concept will be described with reference to the accompanying drawings.

FIGS. 1A to 1C are perspective views of an embodiment of a display device according to the inventive concept. FIG. 1A illustrates a display device in an unfolded state, FIG. 1B illustrates a state in which the display device is folded in a first mode, and FIG. 1C illustrates a state in which the display device is folded in a second mode. The first mode may be an in-folded state, and the second mode may be an out-folded state. Hereinafter, embodiments of the inventive concept will be described with reference to FIGS. 1A to 1C.

Referring to FIG. 1A, a display device DD in an embodiment of the inventive concept may have a quadrangular shape, e.g., rectangular shape having short sides extending in a first direction DR1 and long sides extending in a second direction DR2 crossing the first direction DR1. However, the inventive concept is not limited thereto, and the display device DD may have various shapes such as a circle and a polygon. The display device DD may be a flexible display device.

Hereinafter, a direction substantially perpendicularly crossing a plane defined by the first direction DR1 and the second direction DR2 may be defined as a third direction DR3. In addition, in this specification, the wording “in a plan view” may mean “a state viewed from the third direction DR3.” Hereinafter, the first to third directions DR1 to DR3 are illustrated in the drawings, and the directions indicated by the first to third directions DR1 to DR3 described herein may have a relative concept and may thus be changed to other directions.

The display device DD may include a window WN and a display panel DP. The window WN may be disposed on an upper surface of the display panel DP and may protect the display panel DP. The window WN may be optically transparent, and may have a predetermined rigidity. In this embodiment, the window WN may include glass. The window WN may have a relatively small thickness so as to have flexibility. In an embodiment, the window WN may have a thickness of less than about 100 micrometers (m), for example.

The display panel DP may display an image IM in response to an electrical signal. In this embodiment, the display panel DP may display the image IM on the upper surface, and the window WN may cover the upper surface on which the image IM is substantially displayed. The display panel DP may be a spontaneous light-emitting display panel, and for example, may include an organic light-emitting display panel, an inorganic light-emitting display panel, or a quantum dot light-emitting display panel. However, this is an illustrative embodiment, and the display panel DP may include various display elements such as a reflective display panel as long as the display panel displays an image in response to an electrical signal and has flexibility. The inventive concept is not limited to a particular embodiment.

The display device DD may include a folding region FA and a plurality of non-folding regions NFA1 and NFA2. The non-folding regions NFA1 and NFA2 may include a first non-folding region NFA1 and a second non-folding region NFA2. The folding region FA may be disposed between the first non-folding region NFA1 and the second non-folding region NFA2.

The folding region FA may be a region which overlaps a folding axis FX in a plan view, and a shape of which may be deformed to be folded during folding. In this embodiment, the folding region FA, the first non-folding region NFA1, and the second non-folding region NFA2 may be arranged in the first direction DR1.

By way of example, one folding region FA and two non-folding regions NFA1 and NFA2 are illustrated, but the number of the folding regions FA and the number of the non-folding regions NFA1 and NFA2 are not limited thereto. In an embodiment, the display device DD may include a plurality of non-folding regions, the number of which is more than two, and a plurality of folding regions disposed between the non-folding regions, for example.

A front surface IS of the display device DD may have a flat surface defined by the first direction DR1 and the second direction DR2. In this embodiment, the front surface IS of the display device DD may be a display surface. The display device DD may provide images IM generated in the display device DD to a user through the front surface IS (or display surface).

The front surface IS may include a display region DA and a non-display region NDA around the display region DA. The display region DA may display an image, and the non-display region NDA may not display an image. The non-display region NDA may surround the display region DA, and may define an edge of the display device DD which is printed in a predetermined color.

The display device DD may include at least one sensor SN and at least one camera CA. The sensor SN and the camera CA may be adjacent to the edge of the display device DD. The sensor SN and the camera CA may be disposed in the display region DA adjacent to the non-display region NDA. The sensor SN and the camera CA may be disposed in the second non-folding regions NFA2, but the inventive concept is not limited thereto. The sensor SN and the camera CA may be disposed in the first non-folding regions NFA1.

Light may be transmitted and provided to the sensor SN and the camera CA through parts of the display device DD in which the sensor SN and the camera CA are disposed. In an embodiment, the sensor SN may be a proximity/illuminance sensor, for example, but a type of the sensor SN is not limited thereto. The camera CA may capture an external image. The sensor SN and the camera CA may each be provided in plural.

Referring to FIGS. 1B and 1C, the display device DD may be a folding-type (foldable) display device DD which is folded or unfolded. The folding axis FX may be defined as a major axis in parallel to the long side of the display device DD. In this embodiment, the folding region FA of the display device DD may be folded with respect to the folding axis FX that is parallel to the second direction DR2. However, this is an illustrative embodiment, and the folding axis FX may be set in various directions and set in plurality. The inventive concept is not limited to a particular embodiment.

Referring to FIG. 1B, the display device DD may be in-folded such that the front surface IS is not exposed to the outside. When the display device DD is folded, the first non-folding region NFA1 and the second non-folding region NFA2 may face each other. Here, the front surface IS of the display device DD may be folded so as to wrap around the folding axis FX, and a rear surface RS of the display panel DP of the display device DD may be exposed to the outside.

Referring to FIG. 1C, the display device DD may be out-folded, and when the display device DD is folded, the first non-folding region NFA1 and the second non-folding region NFA2 may be opposed to each other. Here, the rear surface RS (refer to FIG. 1B) of the display device DD may be folded so as to wrap around the folding axis FX, and the front surface IS of the display panel DP of the display device DD may be exposed to the outside.

FIGS. 2A and 2B are perspective views of another embodiment of a display device according to the inventive concept. A front surface IS, a display region DA, a non-display region NDA, a sensor SN and a camera CA, illustrated in FIG. 2A may respectively correspond to the front surface IS, the display region DA, the non-display region NDA, the sensor SN, and the camera CA, illustrated in FIG. 1A. Hereinafter, duplicate explanation will be omitted.

Referring to FIG. 2A, a display device DD′ may have a quadrangular shape, e.g., rectangular shape having long sides extending in a first direction DR1 and short sides extending in a second direction DR2.

A first non-folding region NFA1, a folding region FA, and a second non-folding region NFA2 may be arranged in the first direction DR1. The folding region FA may be disposed between the first non-folding region NFA1 and the second non-folding region NFA2.

Referring to FIGS. 2A and 2B, in the display device DD′, a folding axis FX may be defined as a minor axis in parallel to the short side of the display device DD′. Accordingly, the folding region FA may be bent with respect to the folding axis FX′ that is parallel to the second direction DR2, and thus the display device DD′ may be folded.

In this embodiment, the display device DD′ may include a display module DM and a housing HS. The display module DM may be accommodated in the space provided by the housing HS. The housing HS may include a first housing HS1 and a second housing HS2. The first housing HS1 may overlap the first non-folding region NFA1, and the second housing HS2 may overlap the second non-folding region NFA2. Since the housing HS includes the first housing HS1 and the second housing HS2 which are separated from each other, a folding operation of the display module DM may be readily performed. FIG. 2B illustrates the display device DD′ in an in-folded state. Here, a rear surface HS1_R of the first housing HS1 may be exposed to be viewed from the outside and may protect the display module DM. This is an illustrative embodiment, and the shape and the folding operation of the housing HS may be changed variously. The inventive concept is not limited to a particular embodiment.

FIG. 3 is a cross-sectional view of an embodiment of a display module according to the inventive concept. Referring to FIG. 3, a display module DM may include a display panel DP, an input sensing part SP, an anti-reflection layer RP, and a window WN. This is an illustrative embodiment, and in the display module DM, at least any one of the input sensing part SP or the anti-reflection layer RP may be omitted. In addition to the four components illustrated in FIG. 3, other layers may be added, and the inventive concept is not limited to a particular embodiment.

The display panel DP may be a flexible display panel. The display panel DP in an embodiment of the inventive concept may be a light-emitting display panel, and is not particularly limited. In an embodiment, the display panel DP may be an organic light-emitting display panel or an inorganic light-emitting display panel, for example. A light-emitting layer of the organic light-emitting display panel may include an organic light-emitting material. A light-emitting layer of the inorganic light-emitting display panel may include quantum dots, quantum rods, or the like. Hereinafter, the display panel DP will be described as the organic light-emitting display panel.

The input sensing part SP may be disposed on the display panel DP. The input sensing part SP may include a plurality of sensing parts (not illustrated) for sensing an external input in a capacitive manner. The input sensing part SP may be manufactured directly on the display panel DP during the manufacture of the display module DM. However, the inventive concept is not limited thereto, and the input sensing part SP may be manufactured as a separate panel from the display panel DP, and attached to the display panel DP by an adhesive layer.

The anti-reflection layer RP may be disposed on the input sensing part SP. The anti-reflection layer RP may be directly formed on the input sensing part SP or may be coupled to the input sensing part SP by an adhesive layer. The anti-reflection layer RP may be defined as a film for preventing reflection of external light. The anti-reflection layer RP may reduce reflectance for external light which is incident from outside the display device DD toward the display panel DP through the window WN.

When the external light propagating toward the display panel DP is reflected from the display panel DP and again provided to a user outside the display panel DP, the user may view the external light, as when the display panel is a mirror. To prevent such a phenomenon, the anti-reflection layer RP may include a plurality of color filters for displaying colors which are the same as those of pixels of the display panel DP, for example.

The color filters may filter external light into the same colors as those of the pixels. In such cases, the external light may be invisible to a user. However, the inventive concept is not limited thereto, and the anti-reflection layer RP may include a polarization film for reducing external light reflectance. The polarization film may include a phase retarder and/or a polarizer.

The window WN may be disposed on the anti-reflection layer RP. The window WN may be directly formed on the anti-reflection layer RP or may be coupled to the anti-reflection layer RP by an adhesive layer. The window WN may protect the display panel DP, the input sensing part SP, and the anti-reflection layer RP from an external impact and scratch.

FIG. 4 is a perspective view of an embodiment of a folding inspection apparatus according to the inventive concept.

Referring to FIG. 4, a folding inspection apparatus FD may include a first plate HFR (or upper plate), a second plate LFR (or lower plate), a first fixing member CM1, a second fixing member CM2, a first fixing block FM1, and a second fixing block FM2.

The folding inspection apparatus FD may make the first plate HFR and the second plate LFR spaced apart from each other as distantly as a folded thin-film glass UTG may be disposed (e.g., seated) therebetween. Then, after the folded thin-film glass UTG is disposed (e.g., seated) between the first plate HFR and the second plate LFR, pressure may be applied by making the first plate HFR and the second plate LFR get closer to each other. Here, the first plate HFR may descend at a constant speed until the thin-film glass UTG is broken. The distance between the first plate and the second plate at the time when the thin-film glass UTG is broken may be a flexural strength guaranteeing distance of the thin-film glass UTG.

In the folding inspection apparatus FD according to the inventive concept, the first fixing block FM1 and the second fixing block FM2 may prevent distortion of the thin-film glass UTG, thereby improving the reliability of flexural strength inspection. The folding inspection apparatus FD according to the inventive concept may reduce a process time since a separate inspection of crack lines is omitted by preventing distortion of the thin-film glass UTG.

The thin-film glass UTG which is to be inspected by the folding inspection apparatus FD may include a first portion PUP1 disposed (e.g., seated) on the first plate HFR, a second portion PUP2 disposed (e.g., seated) on the second plate LFR, and a folding portion FUP which is foldable and connected to the first portion PUP1 and the second portion PUP2.

The first plate HFR may have a planar shape in parallel to a plane defined by a first direction DR1 and a second direction DR2. The foldable thin-film glass UTG may be disposed (e.g., seated) on the first plate HFR. The first plate HFR may move along a third direction DR3. After the thin-film glass UTG is disposed (e.g., seated) on the first plate HFR, the first plate HFR may apply pressure to the thin-film glass UTG while the first plate HFR is moving toward the second plate LFR. The first plate HFR may include or consist of a material having durability and rigidity so enough not to be deformed or broken even when the first plate HFR applies pressure to the thin-film glass UTG.

The first portion PUP1 of the thin-film glass UTG may be disposed (e.g., seated) on the first plate HFR. The first portion PUP1 may be disposed on a lower surface of the first plate HFR. Unlike the folding portion FUP, the first portion PUP1 may contact the first plate HFR without being folded. The first portion PUP1 may be parallel to a plane defined by the first direction DR1 and the second direction DR2.

The second plate LFR may be opposed to the first plate HFR in the third direction DR3, and the thin-film glass UTG may be disposed (e.g., seated) thereon. The second plate LFR may have a planar shape in parallel to a plane defined by the first direction DR1 and the second direction DR2. The second plate LFR may be disposed spaced apart from the first plate HFR by a predetermined distance in the third direction DR3. The foldable thin-film glass UTG may be disposed (e.g., seated) on the second plate LFR. The second plate LFR may include or consist of a material having durability and rigidity so enough not to be deformed or broken even when the second plate LFR applies pressure to the thin-film glass UTG.

The second portion PUP2 of the thin-film glass UTG may be disposed (e.g., seated) on the second plate LFR. The second portion PUP2 may be disposed on an upper surface of the second plate LFR. Unlike the folding portion FUP, the second portion PUP2 may contact the second plate LFR without being folded. The second portion PUP2 may be parallel to a plane defined by the first direction DR1 and the second direction DR2.

Since the first plate HFR moves in a direction opposite to the third direction DR3, the distance between the first plate HFR and the second plate LFR may decrease, and pressure may be applied to the thin-film glass UTG. Accordingly, a curvature of the folding portion FUP may increase.

The first fixing member CM1 may be disposed on one end of the first plate HFR, and a plurality of first grooves GR1 may be defined therein. The plurality of first grooves GR1 defined in the first fixing member CM1 will be described later.

The first fixing member CM1 may be disposed at a position corresponding to the second fixing member CM2 in the third direction DR3. The first fixing member CM1 may have a shape extending from the first plate HFR. A width of the first fixing member CM1 in the first direction DR1 may be the same as a width of the first plate HFR in the first direction DR1. A lower surface of the first fixing member CM1 may be smoothly connected to the lower surface of the first plate HFR, and may extend in the second direction DR2.

The first fixing member CM1 may be detachably coupled to one terminal end of the first plate HFR. Accordingly, various types of the first fixing member CM1 may be coupled depending on the size of the thin-film glass UTG, and may fix the thin-film glass UTG in various sizes. Accordingly, folding inspection may proceed regardless of the size of the thin-film glass UTG.

The second fixing member CM2 may be disposed on one end of the second plate LFR, and a plurality of second grooves GR2 may be defined therein. The plurality of second grooves GR2 may respectively correspond to the aforementioned first grooves GR1 in the third direction DR3. The plurality of second grooves GR2 defined in the second fixing member CM2 will be described later.

The second fixing member CM2 may be disposed at a position corresponding to the first fixing member CM1 in the third direction DR3. The second fixing member CM2 may have a shape extending from the second plate LFR. A width of the second fixing member CM2 in the first direction DR1 may be the same as a width of the second plate LFR in the first direction DR1. An upper surface of the second fixing member CM2 may be smoothly connected to the upper surface of the second plate LFR, and may extend in the second direction DR2.

The second fixing member CM2 may be detachably coupled to one terminal end of the second plate LFR. Accordingly, various types of the second fixing member CM2 may be coupled depending on the size of the thin-film glass UTG, and may fix the thin-film glass UTG in various sizes. Accordingly, folding inspection may proceed regardless of the size of the thin-film glass UTG.

The first fixing block FM1 may be coupled to a part of the plurality of first grooves GR1, and may contact at least one outer surface among outer surfaces of the thin-film glass UTG. When the first and second plates HFR and LFR apply pressure to the thin-film glass UTG, the first fixing block FM1 may fix the first portion PUP1 of the thin-film glass UTG such that the position thereof in the second and third directions DR2 and DR3 is not changed. The first fixing block FM1 may fix a terminal end of the first portion PUP1.

The first fixing block FM1 may be coupled to a part of the first grooves GR1, and may protrude in a direction opposite to the third direction DR3. The first fixing block FM1 may be fitted into a part of the first grooves GR1. A first thickness of the first fixing block FM1 along the third direction DR3 may be greater than a thickness of the thin-film glass UTG along the third direction DR3.

When the first fixing block FM1 is coupled to the first grooves GR1, a protruding height in the direction opposite to the third direction DR3 may be greater than the thickness of the thin-film glass UTG. Accordingly, the first fixing block FM1 may more firmly fix the first portion PUP1 of the thin-film glass UTG.

In an embodiment, when the first fixing block FM1 is coupled to the first grooves GR1, and in case where the protruding height in the direction opposite to the third direction DR3 is smaller than the thickness of the thin-film glass UTG, force applied to a portion of the outer surface of the thin-film glass UTG that contacts the first fixing block FM1 may differ from force applied to a portion of the outer surface of the thin-film glass UTG that is not in contact with the first fixing block FM1, for example. Accordingly, distortion of the thin-film glass UTG may occur, and the reliability of flexural strength measurement results may be degraded. When the first fixing block FM1 is coupled to the first grooves GR1, lowering of the reliability of measurement results may be prevented by making the protruding height in the direction opposite to the third direction DR3 greater than the thickness of the thin-film glass UTG.

Since the first fixing block FM1 is desired to fix the thin-film glass UTG at a predetermined position even when pressure is applied to the thin-film glass UTG, the first fixing block FM1 may include or consist of a material having sufficient durability and rigidity. In an embodiment, the first fixing block FM1 may include stainless steel and aluminum, for example.

The first fixing block FM1 may include a (1-1)-th fixing block FM1-1 and a (1-2)-th fixing block FM1-2. The (1-1)-th fixing block FM1-1 may fix the thin-film glass UTG by contacting an outer surface extending in the first direction DR1 among outer surfaces of the first portion PUP1. The (1-2)-th fixing block FM1-2 may fix the thin-film glass UTG by contacting an outer surface extending in the second direction DR2 among the outer surfaces of the first portion PUP1.

The first fixing block FM1 may be detachably coupled to the first fixing member CM1. The first fixing block FM1 may be detachably coupled to a part of the first grooves GR1. The (1-1)-th fixing block FM1-1 may be detachably coupled to (1-1)-th grooves GR1-1 (refer to FIG. 9). The (1-2)-th fixing block FM1-2 may be detachably coupled to (1-2)-th grooves GR1-2 (refer to FIG. 9). That is, the disposition of the first fixing block FM1 may be changed as desired.

The second fixing block FM2 may be coupled to a part of the plurality of second grooves GR2, and may contact at least one outer surface among outer surfaces of the thin-film glass UTG. When the first and second plates HFR and LFR apply pressure to the thin-film glass UTG, the second fixing block FM2 may fix the second portion PUP2 of the thin-film glass UTG such that the position thereof in the second and third directions DR2 and DR3 is not changed. The second fixing block FM2 may fix a terminal end of the second portion PUP2.

The second fixing block FM2 may be coupled to a part of the second grooves GR2, and may protrude in the third direction DR3. The second fixing block FM2 may be fitted into a part of the second grooves GR2. A second thickness of the second fixing block FM2 along the third direction DR3 may be greater than the thickness of the thin-film glass UTG along the third direction DR3.

When the second fixing block FM2 is coupled to the second grooves GR2, a protruding height in the third direction DR3 may be greater than the thickness of the thin-film glass UTG. Accordingly, the second fixing block FM2 may more firmly fix the second portion PUP2 of the thin-film glass UTG.

In an embodiment, when the second fixing block FM2 is coupled to the second grooves GR2, and in case where the protruding height in the third direction DR3 is smaller than the thickness of the thin-film glass UTG, force applied to a portion of the outer surface of the thin-film glass UTG that contacts the second fixing block FM2 may differ from force applied to a portion of the outer surface of the thin-film glass UTG that is not in contact with the second fixing block, for example. Accordingly, distortion of the thin-film glass UTG may occur, and the reliability of flexural strength measurement results may be degraded. When the second fixing block FM2 is coupled to the second grooves GR2, lowering of the reliability of the measurement results may be prevented by making the protruding height in the third direction DR3 greater than the thickness of the thin-film glass UTG.

Since the second fixing block FM2 is desired to fix the thin-film glass UTG at a predetermined position even when pressure is applied to the thin-film glass UTG, the second fixing block FM2 may include or consist of a material having sufficient durability and rigidity. In an embodiment, the second fixing block FM2 may include stainless steel and aluminum, for example.

The second fixing block FM2 may include a (2-1)-th fixing block FM2-1 and a (2-2)-th fixing block FM2-2. The (2-1)-th fixing block FM2-1 may fix the thin-film glass UTG by contacting an outer surface extending in the first direction DR1 among outer surfaces of the second portion PUP2. The (2-2)-th fixing block FM2-2 may fix the thin-film glass UTG by contacting an outer surface extending in the second direction DR2 among the outer surfaces of the second portion PUP2.

FIG. 5 is a perspective view of an embodiment of a folding inspection apparatus according to the inventive concept.

Referring to FIG. 5, a distance DIP between a first plate HFR and a second plate LFR is reduced, and pressure may be applied in a third direction DR3 to a thin-film glass UTG which is disposed (e.g., seated) between the first plate HFR and the second plate LFR. Accordingly, a curvature of a folding portion FUP may become greater. A folding inspection apparatus FD may measure the flexural strength of the thin-film glass UTG by measuring the distance DIP between the first plate HFR and the second plate LFR at the time when the thin-film glass UTG is broken.

A first fixing block FM1 and a second fixing block FM2 may not overlap each other in the third direction DR3. The first fixing block FM1 and the second fixing block FM2 may each be provided in plural, and the plurality of first fixing blocks FM1 and the plurality of second fixing blocks FM2 may be alternately disposed along a first direction DR1. Some first fixing blocks among the plurality of first fixing blocks FM1 and some second fixing blocks among the second fixing blocks FM2 may be alternately disposed along a second direction DR2.

Accordingly, although the first fixing block FM1 and the second fixing block FM2 each have a height greater than the thickness of the thin-film glass UTG, it is possible to prevent the first fixing block FM1 and the second fixing block FM2 from being in contact with each other or interfering with each other when the first plate HFR and the second plate LFR get closer to each other.

FIG. 6 is a front perspective view of an embodiment of a folding inspection apparatus according to the inventive concept.

Referring to FIG. 6, minimum distances DI1 and DI2 along a first direction DR1 between first fixing blocks FM1 and second fixing blocks FM2 may be about 5 mm or more. In case where the minimum distances DI1 and DI2 between the first fixing blocks FM1 and the second fixing blocks FM2 are less than about 5 mm, the first fixing block FM1 and the second fixing block FM2 may be likely to interfere with each other or to contact each other when a first plate HFR and a second plate LFR get closer to each other.

A width WD1 of the first fixing block FM1 along the first direction DR1 and a width WD2 of the second fixing block FM2 along the first direction DR1 may be greater than the minimum distances DI1 and DI2 along the first direction DR1 between the first fixing blocks FM1 and the second fixing blocks FM2.

A thickness TH1 by which the first fixing block FM1 protrudes from a first fixing member CM1 may be greater than a thickness TH3 of a thin-film glass UTG. A thickness TH2 by which the second fixing block FM2 protrudes from a second fixing member CM2 may be greater than the thickness TH3 of the thin-film glass UTG. Here, the thickness of the thin-film glass UTG may be about 100 m or less. When the thickness of the thin-film glass UTG is more than about 100 m, the thin-film glass UTG may not be readily folded, and may thus be unsuitable for an inspection target.

FIG. 7 is a perspective view of an embodiment of a second fixing member and a second plate of a folding inspection apparatus according to the inventive concept.

Referring to FIG. 7, a plurality of second grooves GR2 may include (2-1)-th grooves GR2-1 extending in a first direction DR1 and (2-2)-th grooves GR2-2 extending in a second direction DR2. As illustrated in FIG. 7, some (2-1)-th grooves GR2-1 among the (2-1)-th grooves GR2-1 may be extended to the (2-2)-th grooves GR2-2. Among the (2-1)-th grooves GR2-1, a (2-1)-th groove GR2-1 that is closest to the (2-2)-th groove GR2-2 may be extended to the (2-2)-th groove GR2-2. Accordingly, the (2-1)-th groove GR2-1 and the (2-2)-th groove GR2-2, which are extended to each other, may define a groove bent into an L shape. However, the (2-2)-th groove GR2-2 and some of the (2-1)-th grooves GR2-1 are not necessarily extended to each other, and may be present separately while spaced apart from each other.

The plurality of (2-1)-th grooves GR2-1 may extend in the first direction DR1 and may be arranged along the first direction DR1. The plurality of (2-2)-th grooves GR2-2 may extend in the second direction DR2.

A second fixing block FM2 may be provided in plural, and the plurality of second fixing blocks FM2 may include a (2-1)-th fixing block FM2-1 and a (2-2)-th fixing block FM2-2. The (2-1)-th fixing block FM2-1 may be detachably coupled to the (2-1)-th groove GR2-1. The (2-2)-th fixing block FM2-2 may be detachably coupled to the (2-2)-th groove GR2-2.

The (2-1)-th fixing block FM2-1 may contact a first outer surface ST1 of a second portion PUP2, and thus prevent the second portion PUP2 from moving in the second direction DR2. The (2-2)-th fixing block FM2-2 may contact a second outer surface ST2 of the second portion PUP2, and thus prevent the second portion PUP2 from moving in the first direction DR1.

FIG. 8 is a perspective view of an embodiment of a first fixing member and a first plate of a folding inspection apparatus according to the inventive concept.

Referring to FIG. 8, a plurality of first grooves GR1 may include (1-1)-th grooves GR1-1 extending in a first direction DR1 and (1-2)-th grooves GR1-2 extending in a second direction DR2. As illustrated in FIG. 8, some (1-1)-th grooves GR1-1 among the (1-1)-th grooves GR1-1 may be extended to the (1-2)-th grooves GR1-2. Among the (1-1)-th grooves GR1-1, a (1-1)-th groove GR1-1 that is closest to the (1-2)-th groove GR1-2 may be extended to the (1-2)-th groove GR1-2. Accordingly, the (1-1)-th groove GR1-1 and the (1-2)-th groove GR1-2, which are extended to each other, may define a groove bent into an L shape. However, the (1-2)-th groove GR1-2 and some of the (1-1)-th grooves GR1-1 are not necessarily extended to each other, and may be present separately while spaced apart from each other.

The plurality of (1-1)-th grooves GR1-1 may extend in the first direction DR1, and may be arranged along the first direction DR1. The plurality of (1-2)-th grooves GR1-2 may extend in the second direction DR2.

A first fixing block FM1 may be provided in plural, and the plurality of first fixing blocks FM1 may include a (1-1)-th fixing block FM1-1 and a (1-2)-th fixing block FM1-2. The (1-1)-th fixing block FM1-1 may be detachably coupled to the (1-1)-th groove GR1-1. The (1-2)-th fixing block FM1-2 may be detachably coupled to the (1-2)-th groove GR1-2.

The (1-1)-th fixing block FM1-1 may contact a first outer surface ST1 of a first portion PUP1, and thus prevent the first portion PUP1 from moving in the second direction DR2. The (1-2)-th fixing block FM1-2 may contact a second outer surface ST2 of the first portion PUP1, and thus prevent the first portion PUP1 from moving in the first direction DR1.

Referring to FIGS. 6 to 8, the (1-1)-th grooves GR1-1 may respectively correspond to the (2-1)-th grooves GR2-1 in a third direction DR3. The (1-2)-th grooves GR1-2 may respectively correspond to the (2-2)-th grooves GR2-2 in the third direction DR3.

FIG. 9 is a plan view of an embodiment of a first fixing member and a second fixing member according to the inventive concept. More specifically, FIG. 9 is a plan view illustrating, on one plane, the first fixing member CM1 (refer to FIG. 4) and the second fixing member CM2 (refer to FIG. 4) in FIG. 4.

Referring to FIG. 9, a first direction DR1′, a second direction DR2′, and a third direction DR3′ may indicate relative directions, and may indicate different directions from the aforementioned first, second, and third directions DR1, DR2, and DR3. Here, a first fixing block FM1 coupled to a first groove GR1 and a second fixing block FM2 coupled to a second groove GR2 are illustrated by hatching.

A plurality of (1-1)-th grooves GR1-1 may extend along the second direction DR2′, and may be arranged along the second direction DR2′. A (1-1)-th fixing block FM1-1 may be coupled to a part of the (1-1)-th grooves GR1-1. A plurality of (1-2)-th grooves GR1-2 may extend along the third direction DR3′. (1-2)-th fixing blocks FM1-2 may be couped to the (1-2)-th grooves GR1-2.

A plurality of (2-1)-th grooves GR2-1 may extend along the second direction DR2′, and may be arranged along the second direction DR2′. A (2-1)-th fixing block FM2-1 may be coupled to a part of the (2-1)-th grooves GR2-1. A plurality of (2-2)-th grooves GR2-2 may extend along the third direction DR3′. (2-2)-th fixing blocks FM2-2 may be coupled to the (2-2)-th grooves GR2-2.

Referring to FIGS. 5 and 9, as with FIG. 5, even when the first plate HFR and the second plate LFR get closer to each other, the first fixing block FM1 and the second fixing block FM2 may not overlap each other. The (1-1)-th fixing blocks FM1-1 and the (2-1)-th fixing blocks FM2-1 may be spaced apart from each other by a predetermined distance and arranged to engage with each other, and may not overlap each other. The (1-2)-th fixing blocks FM1-2 and the (2-2)-th fixing blocks FM2-2 may be spaced apart from each other by a predetermined distance and arranged to engage with each other, and may not overlap each other.

FIGS. 10A and 1OB are plan views of another embodiment of a first fixing member and a second fixing member according to the inventive concept.

Referring to FIGS. 10A and 10B, (1-1)-th grooves GR1-1 may be included in a first row HR1 and a second row HR2. The first row HR1 may be a row in which the (1-1)-th grooves GR1-1 are arranged along a second direction DR2′. The second row HR2 may be a row which is spaced apart from the first row HR1 along a third direction DR3′ by a predetermined distance, and in which the (1-1)-th grooves GR1-1 are arranged along the second direction DR2′. In the first row HR1, (1-1a)-th grooves GR1-1a may be arranged along the second direction DR2′. In the second row HR2, (1-1b)-th grooves GR1-1b may be arranged along the second direction DR2′.

(1-2)-th grooves GR1-2 may include (1-2a)-th grooves GR1-2a and (1-2b)-th grooves GR1-2b which are arranged along the third direction DR3′.

(2-1)-th grooves GR2-1 may be included in a third row HR3 and a fourth row HR4. The third row HR3 may be a row in which the (2-1)-th grooves GR2-1 are arranged along the second direction DR2′. The fourth row HR4 may be a row which is spaced apart from the third row HR3 along the third direction DR3′ by a predetermined distance, and in which the (2-1)-th grooves GR2-1 are arranged along the second direction DR2′. In the third row HR3, (2-1a)-th grooves GR2-1a may be arranged along the second direction DR2′. In the fourth row HR4, (2-1b)-th grooves GR2-1b may be arranged along the second direction DR2′.

(2-2)-th grooves GR2-2 may include (2-2a)-th grooves GR2-2a and (2-2b)-th grooves GR2-2b which are arranged along the third direction DR3′.

Referring to FIGS. 10A and 10B, a first groove GR1 and a second groove GR2 to which a first fixing block FM1 and a second fixing block FM2 are respectively coupled may be changed depending on a size of a thin-film glass UTG.

In an embodiment, when the size of the thin-film glass UTG in the third direction DR3′ is relatively great, the first fixing block FM1 may be coupled to the first groove GR1 of the second row HR2, and the second fixing block FM2 may be coupled to the second groove GR2 of the fourth row HR4, as illustrated in FIG. 10A, for example.

When the size of the thin-film glass UTG in the third direction DR3′ is relatively small, the first fixing block FM1 may be coupled to the first groove GR1 of the first row HR1, and the second fixing block FM2 may be coupled to the second groove GR2 of the third row HR3, as illustrated in FIG. 10B.

FIGS. 11A and 11B are plan views of another embodiment of a first fixing member and a second fixing member according to the inventive concept.

Referring to FIGS. 11A and 11B, (1-2)-th grooves GR1-2 may include a (1-2a)-th groove GR1-2a and a (1-2b)-th groove GR1-2b which are arranged in a second direction DR2′. (2-2)-th grooves GR2-2 may include a (2-2a)-th groove GR2-2a and a (2-2b)-th groove GR2-2b which are arranged in the second direction DR2′. The (1-2)-th groove GR1-2 and the (2-2)-th groove GR2-2 to which a first fixing block FM1 and a second fixing block FM2 are respectively coupled may be changed depending on a size of a thin-film glass UTG.

In an embodiment, when the size of the thin-film glass UTG in the second direction DR2′ is relatively great, a (1-2)-th fixing block FM1-2 may be coupled to the (1-2b)-th groove GR1-2b, and a (2-2)-th fixing block FM2-2 may be coupled to the (2-2b)-th groove GR2-2b, as illustrated in FIG. 11A, for example.

When the size of the thin-film glass UTG in the second direction DR2′ is relatively small, the (1-2)-th fixing block FM1-2 may be coupled to the (1-2a)-th groove GR1-2a, and the (2-2)-th fixing block FM2-2 may be coupled to the (2-2a)-th groove GR2-2a, as illustrated in FIG. 11B.

In a display device according to the inventive concept, first and second fixing blocks may be alternately arranged while not overlapping each other, and may not interfere with each other even in a state in which a thin-film glass is folded, so that distortion of the thin-film glass may be prevented.

In the above, description has been made with reference to embodiments, but those skilled in the art or those of ordinary skill in the relevant technical field may understand that various modifications and changes may be made to the inventive concept within the scope not departing from the spirit and the technology scope of the inventive concept described in the claims to be described later. Therefore, the technical scope of the inventive concept is not limited to the contents described in the detailed description of the specification, but should be determined by the claims.

What is claimed is:

1. A folding inspection apparatus comprising:
a first plate on which a foldable thin-film glass is disposed;
a second plate which faces the first plate in a first direction, and on which the foldable thin-film glass is disposed;
a first fixing member which is disposed on an end of the first plate, and in which a plurality of first grooves is defined;
a second fixing member which is disposed on an end of the second plate, and in which a plurality of second grooves respectively corresponding to the plurality of first grooves in the first direction is defined;
a first fixing block coupled to a part of the plurality of first grooves, and contacting at least one outer surface among outer surfaces of the foldable thin-film glass; and
a second fixing block coupled to a part of the plurality of second grooves, and contacting at least one outer surface among the outer surfaces of the foldable thin-film glass,
wherein the first fixing block and the second fixing block do not overlap each other in the first direction.

2. The folding inspection apparatus of claim 1, wherein the first fixing block and the second fixing block are each provided in plural, and
a plurality of first fixing blocks and a plurality of second fixing blocks are alternately disposed in a second direction crossing the first direction and in a third direction crossing the second direction.

3. The folding inspection apparatus of claim 1, wherein a first thickness of the first fixing block along the first direction and a second thickness of the second fixing block along the first direction are greater than a thickness of the foldable thin-film glass along the first direction.

4. The folding inspection apparatus of claim 1, wherein the plurality of first grooves comprises a (1-1)-th groove extending in a second direction crossing the first direction, and a (1-2)-th groove extending in a third direction crossing the second direction,
the plurality of second grooves comprises a (2-1)-th groove extending in the second direction, and a (2-2)-th groove extending in the third direction, and
the (1-1)-th groove corresponds to the (2-1)-th groove in the first direction, and the (1-2)-th groove corresponds to the (2-2)-th groove in the first direction.

5. The folding inspection apparatus of claim 4, wherein the (1-1)-th groove is provided in plural,
at least one (1-1)-th groove of a plurality of (1-1)-th grooves is extended to the (1-2)-th groove,
the (2-1)-th groove is provided in plural, and
at least one (2-1)-th groove of a plurality of (2-1)-th grooves is extended to the (2-2)-th groove.

6. The folding inspection apparatus of claim 4, wherein the (1-1)-th groove and the (2-1)-th groove are each provided in plural,
a plurality of (1-1)-th grooves comprises a first row in which (1-1)-th grooves among the plurality of (1-1)-th grooves are arranged along the second direction, and a second row in which (1-1)-th grooves among the plurality of (1-1)-th grooves are arranged along the second direction and which is spaced apart from the first row along the third direction, and
a plurality of (2-1)-th grooves comprises a third row in which (2-1)-th grooves among the plurality of (2-1)-th grooves are arranged along the second direction, and a fourth row in which (2-1)-th grooves among the plurality of (2-1)-th grooves are arranged along the second direction and which is spaced apart from the third row along the third direction.

7. The folding inspection apparatus of claim 4, wherein the (1-2)-th groove and the (2-2)-th groove are each provided in plural, and
a plurality of (1-2)-th grooves is arranged along the third direction, and a plurality of (2-2)-th grooves is arranged along the third direction.

8. The folding inspection apparatus of claim 4, wherein the first fixing block is provided in plural,
a plurality of first fixing blocks comprises a (1-1)-th fixing block coupled to the (1-1)-th groove, and a (1-2)-th fixing block coupled to the (1-2)-th groove,
the second fixing block is provided in plural, and
a plurality of second fixing blocks comprises a (2-1)-th fixing block coupled to the (2-1)-th groove, and a (2-2)-th fixing block coupled to the (2-2)-th groove.

9. The folding inspection apparatus of claim 8, wherein a minimum distance along the second direction between the plurality of first fixing blocks and the plurality of second fixing blocks is about 5 millimeter or more.

10. The folding inspection apparatus of claim 8, wherein the (1-1)-th fixing block and the (2-1)-th fixing block contact a first outer surface, which extends in the second direction, among the outer surfaces of the foldable thin-film glass, and
the (1-2)-th fixing block and the (2-2)-th fixing block contact a second outer surface, which extends in the third direction, among the outer surfaces of the foldable thin-film glass.

11. The folding inspection apparatus of claim 1, wherein the foldable thin-film glass disposed on the first plate and the second plate includes a folded portion of which a curvature becomes greater as the first plate and the second plate get closer to each other.

12. The folding inspection apparatus of claim 1, wherein the first fixing member corresponds to the second fixing member in the first direction.

13. The folding inspection apparatus of claim 1, wherein the foldable thin-film glass comprises:
a first portion disposed on the first plate;
a second portion disposed on the second plate; and
a folding portion connected to the first portion and the second portion, and foldable,
the first fixing block fixes a terminal end of the first portion, and
the second fixing block fixes a terminal end of the second portion.

14. The folding inspection apparatus of claim 1, wherein a thickness of the foldable thin-film glass is about 100 micrometers or less.

15. The folding inspection apparatus of claim 1, wherein the first fixing member is detachably coupled to the first plate, and
the second fixing member is detachably coupled to the second plate.

16. The folding inspection apparatus of claim 1, wherein the first fixing block is detachably coupled to the first fixing member, and
the second fixing block is detachably coupled to the second fixing member.

17. A folding inspection apparatus comprising:
a first plate on which a foldable thin-film glass is disposed;
a second plate which faces the first plate in a first direction, and on which the foldable thin-film glass is disposed;
a first fixing member which is disposed on an end of the first plate, and in which a plurality of first grooves is defined;
a second fixing member which is disposed on an end of the second plate, and in which a plurality of second grooves respectively corresponding to the plurality of first grooves in the first direction is defined;
a first fixing block coupled to a part of the plurality of first grooves, and contacting at least one outer surface among outer surfaces of the foldable thin-film glass; and
a second fixing block coupled to a part of the plurality of second grooves, and contacting at least one outer surface among the outer surfaces of the foldable thin-film glass,
wherein the first fixing block and the second fixing block are each provided in plural, and a plurality of first fixing blocks and a plurality of second fixing blocks are alternately disposed along a second direction crossing the first direction and along a third direction crossing the second direction.

18. The folding inspection apparatus of claim 17, wherein a first thickness of the first fixing block along the first direction and a second thickness of the second fixing block along the first direction are greater than a thickness of the foldable thin-film glass along the first direction.

19. The folding inspection apparatus of claim 17, wherein a minimum distance along the second direction between the plurality of first fixing blocks and the plurality of second fixing blocks is about 5 millimeters or more.

20. The folding inspection apparatus of claim 17, wherein the foldable thin-film glass comprises:
a first portion disposed on the first plate;
a second portion disposed on the second plate; and
a folding portion connected to the first portion and the second portion, and foldable,
the first fixing block fixes a terminal end of the first portion, and
the second fixing block fixes a terminal end of the second portion.
